(19) United States
(12) Patent Application Publication
(10) Pub. No.: US 2011/0144717 A1
(43) Pub. Date: Jun. 16, 2011

(54) Implantable neurostimulation system and methods of using the system for appetite control and pain control

(75) Inventors: Charles Victor Burton, St. Paul, MN (US); Robert Harold Lovett, Savage, MN (US)

(73) Assignee: Paunceforte Technologies, LLC, Shakopee, MN (US)

(21) Appl. No.: 12/635,266

(22) Filed: Dec. 10, 2009

(51) Int. Cl. A61N 1/34; A61N 1/08

(52) U.S. Cl. 607/46; 607/60

(57) ABSTRACT

An implantable electronic device includes a geodesic shaped dome housing and means for attaching the dome to a target neurologic structure. The dome includes a radio frequency receiver, an amplifier, and a stimulating electrode. A radio frequency based neurostimulatory system further includes a transmitting coil positioned outside a patient's body for transmitting pulses to the receiver and activating the stimulating electrode within the implantable electronic device. A method of stimulating a patient's neurologic structure includes implanting an electronic device proximate the neurologic structure, positioning a transmitting coil outside the patient's body for controlling the implantable device, initiating radio frequency waves from a pulse generator to the transmitting coil which in turn activates the stimulating electrode. The neurostimulatory system can be used for relief of visceral and somatic pain as well as for controlling appetite in patients.

### FIELD OF THE INVENTION

The present invention relates to a neurostimulation system that includes an implantable electronic device. This system is particularly useful for the purpose of appetite control, as well as for pain control of visceral and somatic origin.

### BACKGROUND OF THE INVENTION

Control of somatic pain through the implantation of neurostimulating electrodes has been a well-established therapeutic procedure since the late 1970s. To date however there has been no effective means of visceral pain control by neurostimulation. There is presently no effective long-term treatment for the cruel and incapacitating visceral pain of cancers, such as that of the pancreas, other than by high doses of narcotic drugs.

Stimulation of the vagus nerves, and its branches, has been initiated as a means of appetite suppression and as an alternative to bariatic surgery. Transcutaneous electrical nerve stimulation (TENS) is described in U.S. Pat. No. 7,200,443 to reduce eating and for gastrointestinal disorders. This approach utilizes a small electrical device to deliver electrical impulses through the skin via electrode pads affixed externally to the skin in the thoracic region of the spine at the level of T6-T10.

Wire leads have been used as a means of reducing somatic pain or appetite suppression but there is a high risk of associated device lead wire failure rate due to the hostile environment of the human body. While this is true for all neurologic targets, it is particularly true in regard to the remote location of the celiac plexus. Radio frequency coupled neurostimulators have been in use for various applications for over 30 years. In all such cases, the transmitting antenna is placed on the skin and the radio frequency receiver is placed subcutaneously close to the transmitting device in order to couple with it.

### SUMMARY OF THE INVENTION

In a first aspect, the present invention relates to a radio frequency based system for stimulating a target structure in a patient. The device includes an implantable electronic device comprising a housing, wherein the housing comprises a radio frequency signal receiver, an amplifier and a stimulating electrode. The receiver can receive signals in a three-dimensional manner when implanted in the target structure of the patient's body. The system also includes a transmitting coil placed on the exterior surface of the patient's body for transmitting a signal to the receiver of the implantable electronic device. The housing of the device can include a geodesic shaped dome.

In another aspect, the present invention includes an implantable electronic device comprising a housing, wherein the housing comprises a radio frequency receiver, an amplifier and a stimulating electrode. The device also includes a means for attaching the housing to a target structure within a patient.

In yet another aspect, the present invention includes a method of stimulating a patient's neurologic structure. The method includes implanting an electronic device comprising a housing, wherein the housing comprises a radio frequency signal receiver, an amplifier and a stimulating electrode, wherein the receiver can receive signals in a three-dimensional manner when implanted in the target structure of the patient's body. The method also includes positioning a transmitting coil on the exterior side of the patient's body for controlling the stimulating electrode of the electronic device. The method further includes initiating radio frequency waves from a pulse generator to the transmitting coil, wherein the transmitting coil transmits radio frequency waves to the electronic device to stimulate the neurologic structure.

In a further aspect, the present invention includes a method of controlling pain in a patient. The method includes implanting an electronic device comprising a housing, wherein the housing comprises a radio frequency signal receiver, an amplifier and a stimulating electrode, wherein the receiver can receive signals in a three-dimensional manner when implanted on the celiac plexus of the patient. The method also includes positioning a transmitting coil on the exterior side of the patient's body for controlling the stimulating electrode of the electronic device. The method further includes initiating radio frequency waves from a pulse generator to the transmitting coil, wherein the transmitting coil transmits radio frequency waves to the electronic device to stimulate the celiac plexus.

In yet a further aspect, the present invention includes a method of controlling appetite in a patient. The method includes implanting an electronic device comprising a housing, wherein the housing comprises a radio frequency signal receiver, an amplifier and a stimulating electrode, wherein the receiver can receive signals in a three-dimensional manner when implanted on the celiac plexus of the patient. The method includes positioning a transmitting coil on the exterior side of the patient's body for controlling the stimulating electrode of the electronic device. The method further includes initiating radio frequency waves from a pulse generator to the transmitting coil, wherein the transmitting coil transmits radio frequency waves to the electronic device to stimulate the celiac plexus.

### BRIEF DESCRIPTION OF THE DRAWINGS

FIG. 1a is a schematic diagram of the implantable electronic device.

FIG. 1b is a representation of the implantable electronic device.

FIG. 2 shows the implantation of the device by an endoscope.

FIG. 3 shows the internally placed device affixed to the surface of the celiac plexus.

FIG. 4 is a representation of the external pulse generator controlled by the patient.

### DETAILED DESCRIPTION OF ILLUSTRATIVE EMBODIMENTS

The present invention relates to a radio frequency based stimulation system that can be used to stimulate target structures in a patient, particularly neurological targets. This neurostimulation system includes an implantable electronic device, an external transmitting coil and a pulse generator. The implantable electronic device houses multiple components including a radio frequency receiver, an amplifier and a stimulating electrode and, in addition, the device is structured to receive signals in a three-dimensional manner. In a preferred embodiment, the components of the implantable device are housed in a geodesic shaped dome structure. The geodesic shaped dome can be implanted in a patient at a desired target neurologic area or structure.

The neurostimulation system also includes a transmitting coil that is placed externally on the patient. The transmitting coil receives a signal from a pulse generator that is external to the patient's body. The transmitting coil can transmit radio frequency waves to the receiver in the implantable device. The receiver can capture the signal and ultimately activate the stimulating electrode within the implantable device. The activated stimulating electrode stimulates the structure on which it is located.

A geodesic shaped dome, as is commonly known, is a spherical or partial-spherical shell structure or lattice shell based on a network of great circles (geodesics) lying on the surface of a sphere. The geodesics intersect to form triangular elements that have local triangular rigidity and also distribute the stress across the entire structure. Geodesic shaped dome as used herein can refer to the implantable electronic device and include, for example, the multiple components described herein. However, housing structures of other shapes may also be used and are also within the scope of the invention.

The present invention also includes a method for using the neurostimulation system to stimulate neurological structures such as the celiac plexus. In a preferred embodiment, a minimally invasive endoscopic surgical procedure is used for implanting the geodesic shaped dome housing that includes the receiver, amplifier, and stimulating electrode. The geodesic shaped dome housing may be placed upon or within the celiac plexus or other target neurologic structures. Electrical stimulation of the celiac plexus can be effective both for visceral pain control as well as for appetite control, depending on the stimulation parameters. The celiac plexus can be stimulated by the internal device when communicating with an external radio frequency wave transmitting coil to control the patient symptoms. This novel type of neurostimulator has been specifically designed to be used for these purposes, as well as a new means of controlling visceral as well as somatic pain.

The neurostimulator system described herein advantageously avoids the pitfalls of previous direct and indirect neurostimulator systems. Prior art radio frequency stimulators have used a two dimensional system for receiving signals. In contrast, the present invention receives signals in a three-dimensional or multi-directional manner leading to a more effective means of stimulating structures. With the remote system described herein, the user has complete external control of the system. The implanted device itself is inert until activated by the external radio frequency pulse generator. The present system can also allow stimulation of structures deep within the body as opposed to structures immediately beneath the skin surface. The neurostimulator system described herein can also minimize side effects through providing the most direct, discrete and local stimulation. In addition, the system and the methods can provide treatment of obesity and control of visceral pain and somatic pain in a user-friendly manner.

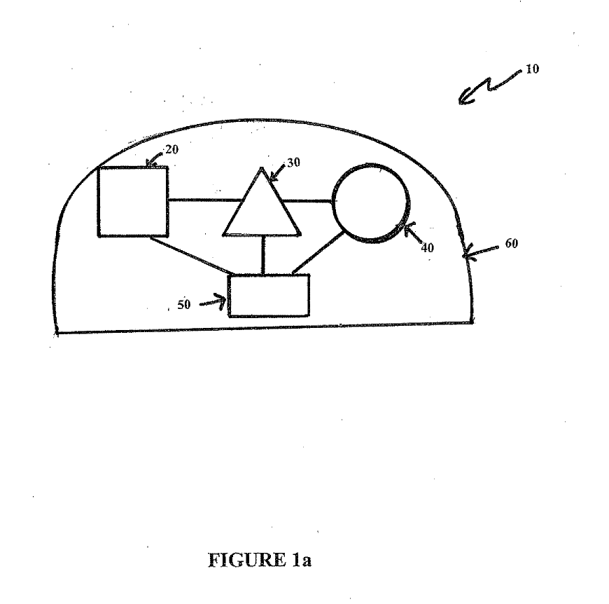

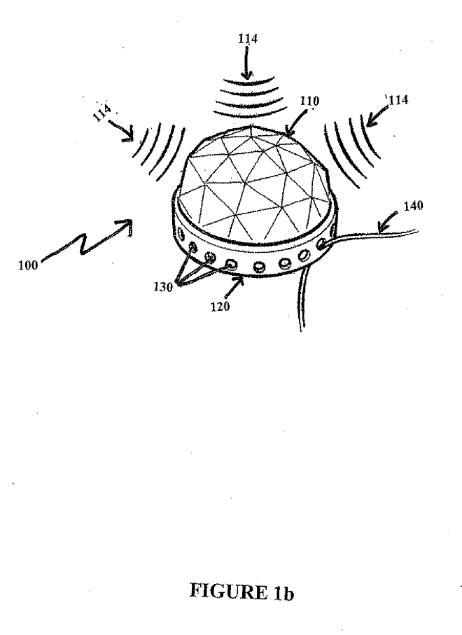

FIG. 1a shows a schematic diagram of an embodiment of the implantable device of the present invention. Implantable device 10 includes radio frequency receiver 20, amplifier 30 and stimulating electrode 40. Generally, receiver 20 is electrically connected to amplifier 30 which in turn is electrically connected to electrode 40. The device 10 preferably also includes a power source 50 that is connected to receiver 20, amplifier 30 and electrode 40. Power source 50 can be, for example, a battery encased in a substance such as titanium. Other power sources are also within the scope of this invention. Housing 60 contains all of the components of device 10. Housing 60 of the implantable device is designed to capture radio frequency pulses in a three-dimensional manner directed toward it by an external radio frequency power source. A variety of types and shapes of housing are within the scope of this invention.

FIG. 1b shows geodesic shaped dome 100 which is one preferred embodiment for housing of an implantable device. Geodesic shaped dome can be made from a variety of materials, preferably biocompatible materials. One exemplary embodiment includes geodesic shaped dome made from non-metallic, ceramic material. Geodesic shaped dome 100 houses, for example, the components shown in FIG. 1a and can receive pulses 114 in a three-dimensional manner. Geodesic shaped dome 100 includes dome 110 and suture skirt 120. Suture skirt 120 preferably includes apertures 130 such that suture 140 can pass through the apertures. The diameter of suture skirt 120 can vary but generally is approximately similar to the diameter of dome 100. Suture skirts are well known in the art and can be made from a variety of materials including natural and/or synthetic materials that are biocompatible. Alternatively, the device can be affixed with an adhesive or a crimping device.

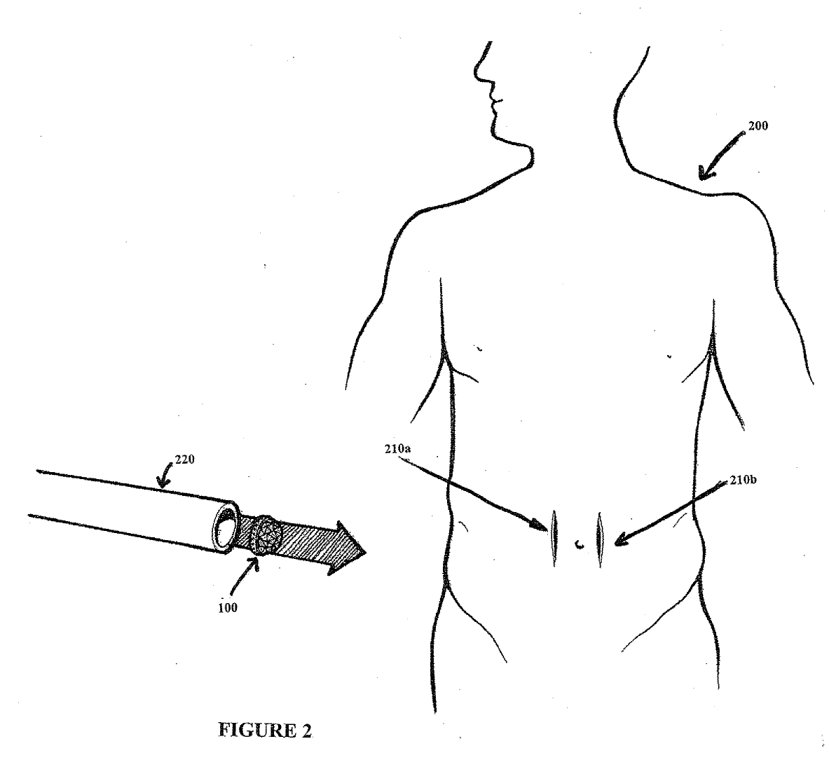

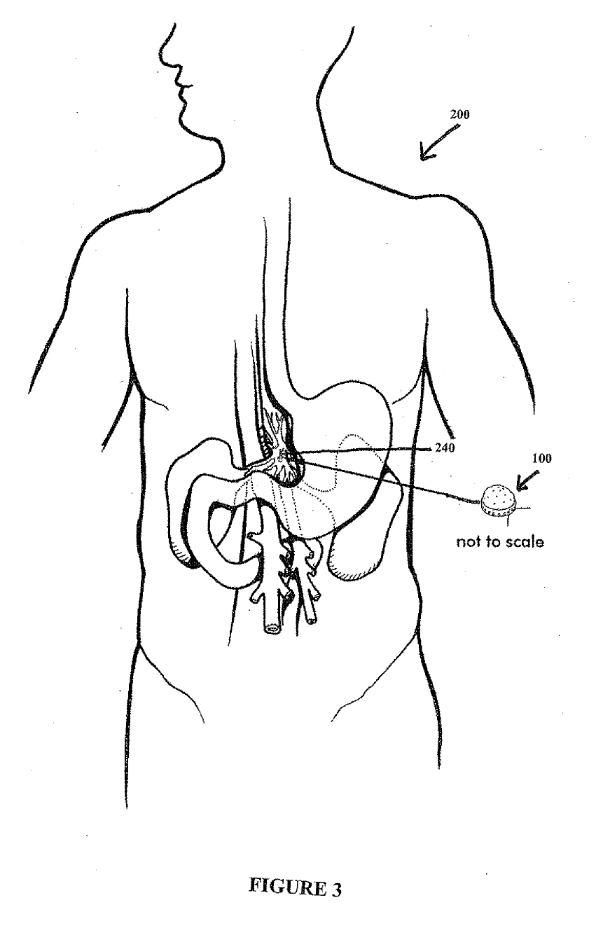

FIG. 2 shows patient 200 with two small incisions, 210a and 210b. In a preferred method, endoscope 220 is used to implant geodesic shaped dome 100 into patient 200 via incisions 210a and 210b. Geodesic shaped dome 100 is sized appropriately to fit within endoscope 220 to enable insertion into patient 200. The insertion is preferably in the patient's flanks. FIG. 3 illustrates the placement of geodesic shaped dome 100 in celiac plexus 240 of patient 200. Geodesic shaped dome 100 is oriented toward the outside of the patient's body.

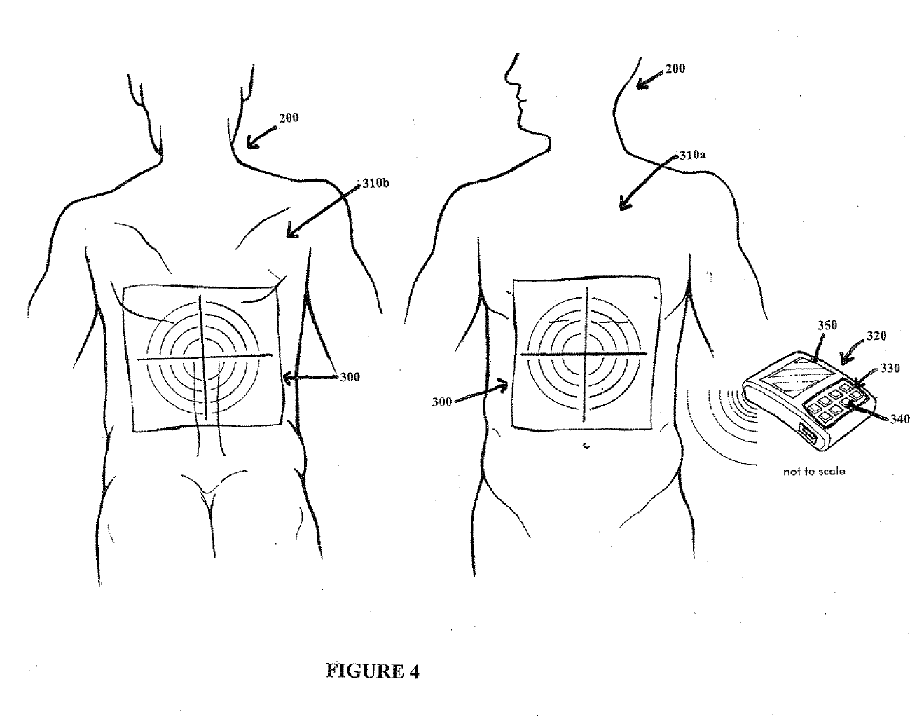

The present invention also includes an external transmitting coil and a pulse generator. The external transmitting coil is generally placed on the exterior of the patient, preferably on the backside or the frontside of the patient's thoraco-lumbar spine. The transmitting coil may be placed, for example, as an appliqué. The pulse generator is external to the patient's body, i.e. not attached to the body. FIG. 4 schematically illustrate the placement of transmitting coil 300 on either the front side 310a or backside 310b of patient 200. More than one transmitting coil 300 may be placed on a patient. In embodiments containing more than one transmitting coil, the coils are constructed to provide different stimulation parameters so that there is no interference or cancellation of the signal. In FIG. 4, pulse generator 320 is a small device that includes keypad 330 with keys 340. The pulse generator optionally includes display screen 350. The pulse generator can be handheld or placed on a table and can be operated by the patient, another operator or computer controlled.

As shown in FIGS. 2 and 3, the geodesic shaped dome can be surgically placed through an endoscope and affixed directly to neurologic targets such as the celiac plexus. The use of an endoscope is advantageous because it is minimally invasive and allows quick recovery of the patient with fewer complications. Other techniques such as regular surgical procedures may be used and are within the scope of the invention. The geodesic shaped dome can be held in place by sutures through the suture skirt that preferably is attached to the dome. Other attachment means, such as using adhesives or claw like crimping structures at the bottom of the dome that hold the geodesic shaped dome in place are also within the scope of the invention. The orientation of the transmitting coil can vary and the geodesic shaped dome housing the receiver can facilitate a number of different transmitting coil designs.

The transmitting coil may send signals to the geodesic shaped dome in a continuous manner, periodic manner and/or in a defined manner that is determined by the patient, physician or another operator. A variety of parameters can be manipulated and can determine the type of response a patient exhibits. These parameters include the length or duration of the signal, the amplitude or intensity of the signal, and the periodicity at which the radio waves are emitted.

The pulse generator may be controlled manually. When a signal is desired, the operator can for example, set the intensity, periodicity and duration of the signal on the pulse generator and manually operate the pulse generator so the desired radio frequency waves are emitted to the transmitting coil which in turn transmits the signal to the geodesic shaped dome. The pulse generator may also be a programmable device that is programmed for future transmissions based on historical data recorded in the pulse generator. The pulse generator can also be adaptable to be connected to a computer that can then be used to download information into the pulse generator for determining the transmission parameters and/or schedule. Some pulse generators may have a combination or all of these features.

In one preferred embodiment, the pulse generator includes both a manual and a programmable mode. In the manual mode, the pulse generator is controlled manually as the patient begins to notice hunger pains. The patient controls the frequency and temporal occurrences of the transmissions as well as the parameters of the transmissions to the receiver using the keyboard built into the pulse generator. The pulse generator can record and display the time and date and parameters of the previous doses for reference by the patient to assist in the decision making and to avoid under or over dosing. The pulse generator may also include a pre-settable alarm that can be heard audibly or vibrate, in the manner of a cell phone to remind the patient to administer a dose. This alarm can also remind the patient in the event a dose has not been taken within the time set by the patient.

In the programmable mode, the alarm can sound prior to a programmed dose, so as to permit the patient to override the programming and return to the manual mode. In the programmed iteration, the programming may take place on a connected desktop or laptop computer or using the keyboard. The pulse generator can communicate with a desktop or laptop computer via a USB port. Over time, when the patient becomes aware of the ideal frequency, timing, and strength, the patient may elect to replay a course of treatment from a previous period of time with such modifications as the patient wishes to program. To facilitate programming, the pulse generator may contain a USB port to communicate with the patient's home computer, permitting the patient to view prior history and set future programs. When plugged in with the USB cable, the pulse generator's re-chargeable battery may recharge. The device may also include a battery re-charger for use while the patient is sleeping.

The keyboard may also permit the manual recordation by the patient of the estimated caloric intake using the keyboard built into the pulse generator. This information can be displayed along with the information relating the frequency, timing, and parameters of the transmissions to the internal receiver. All of this information allows the patient and/or healthcare professional to fine-tune the stimulation regimen.

The present invention includes a method of stimulating structures, preferably neurological structures in a patient using the neurostimulatory systems described herein. The patient can be human or animal. A variety of neurological structures can be stimulated including for example, cervical plexus, celiac plexus, brachial plexus, sacral plexus, lumbar plexus and the like. In a preferred embodiment, the celiac plexus is stimulated. The method includes placing the implantable device in, on or within a neurologic structure of the patient. The device may be placed using a variety of techniques including by the use of an endoscope. Preferably, the device is secured in the target structure by any of the attachment means described above. A transmitting coil can be attached to the patient who has the implantable device in place. The transmitting coil can be, for example, in the form of an appliqué patch. Alternatively, the transmitting coil can be attached to or within another structure such as an ace bandage and the like.

When stimulation of the target structure is desired, a pulse can be initiated from the pulse generator. The pulse generator can be handheld or on a tabletop but it is close enough to the patient that the pulse generator can send the signal to the transmitting coil to emit the radio frequency waves to the device within the patient. The periodic or continual stimulation of the receiver provides impulses to the target area. The pulse generator that initiates the pulses that ultimately lead to stimulation of the target structure may be operated manually or programmed as described above to administer a desired stimulation regimen to the patient.

The present invention preferably relates to stimulating the celiac plexus in a patient. Stimulation of the celiac plexus can lead to appetite suppression and/or pain control depending on the specific stimulation parameters used. In some embodiments, the celiac plexus is stimulated by low level electric pulses which in turn can control appetite by inducing low grade nausea but not at the expense of undue comfort to the patient. The low grade nausea can suppress the patient's appetite and lead to weight loss. The celiac plexus when stimulated with different stimulation parameters or intensities of electric pulses can control visceral and/or somatic pain. Generally, the level of the transmission can be initially adjusted by the healthcare professional and the frequency of the doses can be monitored and optimized during an initial course of treatment, but with the goal of essentially achieving self-control by the patient of the device.

Stimulation parameters used for a patient can vary based on the goal of the therapy, i.e. appetite suppression, pain relief and/or pain control. Stimulation parameters can also vary from patient to patient. Stimulation parameters that may produce pain control in one patient may produce pain relief in another patient. Generally, the stimulation parameters are optimized for each individual patient. A patient's body habitus can play an important role in the stimulation parameters used for a particular desired result. A patient's innate pain tolerance can also be seminal to the stimulation parameters selected.

The neurologic structure can be stimulated at a variety of intensities. The neurological structure is generally stimulated between about 20 Hz and about1,000 Hz. Preferably, the neurological structure is stimulated at between about 50 Hz and about 200 Hz. Stimulation with intensities outside of these ranges are also within the scope of this invention.

The neurologic structure can be stimulated for varying lengths of time at varying periodicities. In one exemplary embodiment, the neurologic structure may be continually stimulated at a very low grade intensity. In another exemplary embodiment, the neurologic structure may be stimulated for an hour and off for a few hours. In exemplary embodiments for appetite suppression, the neurologic structure may be stimulated prior to mealtimes. Duration and periodicity other than those specifically described herein are also within the scope of this invention.

The present invention can also include a method of controlling pain in a patient. The pain can be a visceral pain as experienced, for example, by patients suffering from pancreatic cancers and the like. The pain can also be somatic pain. The method includes stimulating the celiac plexus of the patient using the neurostimulatory devices described herein. The stimulation regimen for controlling pain in a patient can vary depending on the patient's age, size, health status, the stage of the disease causing the pain, the amount of pain and the like.

The present invention can also include a method of controlling appetite in a patient. The method can result in appetite suppression that results in weight loss for a patient. The method includes stimulating the celiac plexus of the patient using the neurostimulatory devices described herein. The stimulation regimen for controlling appetite in a patient can vary depending on the patient's weight, gender, age, health status and the like.

Although the present invention has been described with reference to preferred embodiments, workers skilled in the art will recognize that changes may be made in form and detail without departing from the spirit and scope of the invention.

What is claimed is:

1. A radio frequency based system for stimulating a target structure in a patient comprising:
an implantable electronic device comprising a housing, wherein the housing comprises a radio frequency signal receiver, an amplifier and a stimulating electrode, wherein the receiver can receive signals in a three-dimensional manner when implanted in the target structure of the patient's body; and
a transmitting coil for placing on the exterior surface of the patient's body for transmitting a signal to the receiver of the implantable electronic device.

2. The system of claim 1 wherein the housing of the implantable electronic device is a geodesic shaped dome.

3. The system of claim 1 further comprising a pulse generator, wherein the pulse generator initiates radio frequency waves toward the transmitting coil.

4. The system of claim 3 wherein the transmitting coil transmits the radio frequency waves from the pulse generator to the radio frequency receiver.

5. The system of claim 3 wherein the pulse generator is programmable.

6. The system of claim 3 wherein the pulse generator is operated by the patient.

7. The system of claim 1 wherein the target structure is celiac plexus.

8. The system of claim 1 wherein the implantable device further comprises a battery.

9. The system of claim 1 wherein the implantable device further comprises an attachment means for attaching the device to the target structure.

10. The system of claim 9 wherein the attachment means is a suture skirt.

11. An implantable electronic device comprising a housing, wherein the housing comprises a radio frequency receiver, an amplifier and a stimulating electrode, the device further comprising a means for attaching the housing to a target structure within a patient.

12. The implantable device of claim 11 wherein the housing is a geodesic shaped dome.

13. The device of claim 11 wherein the means for attaching is a suturing skirt comprising a plurality of apertures.

14. The device of claim 11 wherein the attachment means is an adhesive on the underside of the housing.

15. The device of claim 11 further comprising a battery.

16. The device of claim 11 wherein the receiver can receive a radio frequency signal from a transmitting coil on the exterior side of the patient.

17. A method of stimulating a patient's neurologic structure comprising:
implanting an electronic device comprising a housing, wherein the housing comprises a radio frequency signal receiver, an amplifier and a stimulating electrode, wherein the receiver can receive signals in a three-dimensional manner when implanted in the target structure of the patient's body;
positioning a transmitting coil on the exterior side of the patient's body for controlling the stimulating electrode of the electronic device; and
initiating radio frequency waves from a pulse generator to the transmitting coil, wherein the transmitting coil transmits radio frequency waves to the electronic device to stimulate the neurologic structure.

18. The method of claim 17 wherein the neurologic structure is celiac plexus.

19. The method of claim 17 wherein implanting the electronic device comprises using endoscopic surgery.

20. The method of claim 17 wherein the initiating of the radio frequency waves is performed manually by an operator operating the pulse generator.

21. The method of claim 20 wherein the operator is the patient.

22. The method of claim 17 wherein the initiating of the radio frequency waves is initiated by a programmable pulse generator.

23. A method of controlling pain in a patient comprising:
implanting an electronic device comprising a housing, wherein the housing comprises a radio frequency signal receiver, an amplifier and a stimulating electrode, wherein the receiver can receive signals in a three-dimensional manner when implanted on the celiac plexus of the patient;
positioning a transmitting coil on the exterior side of the patient's body for controlling the stimulating electrode of the electronic device; and
initiating radio frequency waves from a pulse generator to the transmitting coil, wherein the transmitting coil transmits radio frequency waves to the electronic device to stimulate the celiac plexus.

24. The method of claim 23 wherein the pain is a visceral pain.

25. The method of claim 23 wherein the patient is diagnosed with pancreatic cancer.

26. The method of claim 23 wherein the pain is a somatic pain.

27. The method of claim 23 wherein the radio frequency waves are between about 20 Hz and about 1000 Hz.

28. The method of claim 23 wherein the pulse generator is operated by an operator.

29. The method of claim 28 wherein the operator is the patient.

30. The method of claim 23 wherein the pulse generator is programmable to deliver radio frequency waves at predetermined time and settings.

31. A method of controlling appetite in a patient comprising:
implanting an electronic device comprising a housing, wherein the housing comprises a radio frequency signal receiver, an amplifier and a stimulating electrode, wherein the receiver can receive signals in a three-dimensional manner when implanted on the celiac plexus of the patient;
positioning a transmitting coil on the exterior side of the patient's body for controlling the stimulating electrode of the electronic device; and
initiating radio frequency waves from a pulse generator to the transmitting coil, wherein the transmitting coil transmits radio frequency waves to the electronic device to stimulate the celiac plexus.

32. The method of claim 31 wherein the appetite is suppressed.

33. The method of claim 31 wherein the radio frequency waves are between about 20 Hz and about 1000 Hz.

34. The method of claim 31 wherein the pulse generator is operated by an operator.

35. The method of claim 34 wherein the operator is the patient.

36. The method of claim 31 wherein the pulse generator is programmable to deliver radio frequency waves at predetermined time and settings.
